(19) United States
(12) Patent Application Publication
(10) Pub. No.: US 2008/0193522 A1
(43) Pub. Date: Aug. 14, 2008

(54) Use of Polymer Mixtures For the Production of Coated Pharmaceutical Formulations and Pharmaceutical Formulation With Mixed Polymeric Coating

(75) Inventors: Christian Meier, Darmstadt (DE); Karin Knuppen, Steinbach (DE); Hans-Ulrich Petereit, Darmstadt (DE)

Correspondence Address: OBLON, SPIVAK, MCCLELLAND MAIER & NEUSTADT, P.C., 1940 DUKE STREET, ALEXANDRIA, VA, 22314, US

(73) Assignee: ROEHM GMBH, Darmstadt (DE)

(21) Appl. No.: 11/909,315

(22) PCT Filed: Mar. 3, 2006
(86) PCT No.: PCT/EP2006/001949

§ 371 Date: Sep. 21, 2007

(30) Foreign Application Priority Data
May 25, 2005 (DE) 10 2005 024 614.1

(51) Int. Cl. A61K 9/62; A61K 9/00; A61K 9/36; A61K 9/24; A61K 9/30; A61K 9/48

(52) U.S. Cl. 424/461; 424/400; 424/479; 424/475; 424/463; 424/472

(57) ABSTRACT

The invention relates to the use of mixture of 2 to 60 wt. % of one or more polymers (I) with 40 to 98 wt. % of one or more polymers (II), whereby the polymer (I) is a (meth)acrylate copolymer, containing 90 to 100 wt. % radically polymerised of 40 to 95 wt. % of C₁ to C₄ alkyl esters of acrylic or methacrylic acid and 5 to 60 wt. % of units of (meth)acrylate monomers with an anionic group with 0 to 10 wt. % of further vinylic polymerisable monomers and polymer(ll) is a vinyl polymer different from polymer (I) or a polysaccharide or a derivative of a polysaccharide, containing 88 to 100 % neutral monomer units and up to 12 wt. % polymerisable monomer units with ionic groups, for production of a coated pharmaceutical formulation, containing an active agent core and a polymeric coating made from the mixture of polymers (I) and (II), characterised in that the glass temperature of polymer (I) is not more than 70° C. and an active agent release profile is achieved, whereby the agent release is delayed with relation to a pharmaceutical formulation with a coating made exclusively of polymer (I), starting with the same pH. The invention further relates to a pharmaceutical formulation with a selected polymer (I).

The invention relates to the use of polymer mixtures for the production of coated pharmaceutical forms, and to a pharmaceutical form with mixed polymeric coating.

### PRIOR ART

The use of so-called neutral methacrylate copolymers, meaning methacrylate copolymers which consist predominantly of (at least 95%) (meth)acrylate monomers having neutral radicals, such as methyl methacrylate or ethyl acrylate, as coating agents and binders for pharmaceutical forms with delayed released of active ingredient has been known for a long time. Uses in mixtures with anionic dispersions are described for example in EP-A 152 038, EP-A 208 213 or EP-A 617 972. WO 01/68767 describes the production of dispersions comprising neutral methyl acrylate copolymers using 1-10% by weight of a nonionic emulsifier having an HLB of from 15.2 to 17.3. These measures allow the production therefrom, while retaining the stability of the dispersion and its particle size distribution, of pharmaceutical formulations in which phase separation with formation of crystal structures is suppressed by the emulsifier.

EP 0 152 038 A2 describes coated pharmaceutical forms with mixed coatings of water-soluble carboxyl group-containing polymers and water-insoluble, film-forming polymers. The polymers may be present in ratios from 60:40 to 5:95. For example, mixed coatings of polymers which may consist on the one hand of equal parts of ethyl acrylate and methacrylic acid and on the other hand of polymers which are composed of ethyl acrylate and methyl methacrylate in the ratio 2 to 1 are described.

EP 0 208 213 A1 is nearly identical in content to EP 0 152 038 A2, but additionally discloses the effect of high extensibility and elasticity of corresponding mixed coatings.

EP 0 704 208 A2 describes coating agents and binders for pharmaceutical coatings soluble in intestinal juice. These are copolymers of 10 to 25% by weight methacrylic acid, 40 to 70% by weight methyl acrylate and 20 to 40% by weight methyl methacrylate. The description mentions not only monolayer coatings but also multilayer coating systems. The latter may consist of a core, which comprises for example a basic or a water-sensitive active ingredient, have a sealing layer of a different coating material such as cellulose ether, cellulose ester or a cationic polymethacrylate, e.g. of the EUDRAGIT® type, inter alia including EUDRAGIT® RS and RL, and are then additionally provided with the abovementioned coating soluble in intestinal juice.

WO 03/072087 describes a process for producing a pharmaceutical form in which there is use of a copolymer which is composed of
20 to 34% by weight methacrylic acid and/or acrylic acid,
20 to 69% by weight methyl acrylate and
0 to 40% by weight ethyl acrylate and/or optionally
0 to 10% by weight further vinylically copolymerizable monomers,
   with the proviso that the glass transition temperature of the copolymer in accordance with ISO 11357-2, subclause 3.3.3, is not more than 60° C.

It may be advantageous in the individual case for controlling the delivery of active ingredient to admix further polymers with the copolymer. The proportion of further polymers in the mixture may vary within wide limits and is between 1 and 99%, preferably between 10 and 90% by weight, particularly preferably between 25 and 85% by weight, based on the polymer mixture.

Examples of such further polymers are: polyvinyl-pyrrolidones, polyvinyl alcohols, anionic (meth)acrylate copolymers of methyl methacrylate and/or ethyl acrylate and methacrylic acid (EUDRAGIT® L 100, EUDRAGIT® S 100, EUDRAGIT® L 100-55). Anionic (meth)acrylate copolymers of methyl methacrylate, methyl acrylate and methacrylic acid of the prior art (see, for example, EP-A 0 704 207 or EP-A 0 704 208), carboxymethylcellulose salts, hydroxypropylcellulose (HPMC), neutral (meth)acrylate copolymers of methyl methacrylate and ethyl acrylate (dry matter from EUDRAGIT® NE 30 D), copolymers of methyl methacrylate and butyl methacrylate (PLASTOID® B) or (meth)acrylate copolymers having quaternary ammonium groups (EUDRAGIT® RL and EUDRAGIT® RS).

WO 2004/096185 describes a process for the production of a coated pharmaceutical form or of a pharmaceutical form in the form of an active ingredient-containing matrix, by processing a copolymer, an active pharmaceutical ingredient, a core which is present where appropriate, and/or pharmaceutically customary additives in a manner known per se by melting, injection molding, extrusion, wet granulation, casting, dipping, spreading, spraying or compressing to give a coated pharmaceutical form and/or to give an active ingredient-containing matrix, employing a copolymer which is composed of
20 to 33% by weight methacrylic acid and/or acrylic acid,
5 to 30% by weight methyl acrylate and
20 to 40% by weight ethyl acrylate and more than 10 to 30% by weight butyl methacrylate and optionally
0 to 10% by weight further vinylically copolymerizable monomers, where the proportions of the monomers add up to 100% by weight,
   with the proviso that the glass transition temperature of the copolymer is 55 to 70° C.

It may be advantageous in the individual case for controlling the delivery of active ingredient to admix further polymers with the copolymer. The proportion of further polymers in the mixture may vary within wide limits and is between 5 and 95%, preferably between 10 and 90% by weight, particularly preferably between 25 and 85% by weight.

Examples of such further polymers are: polyvinyl-pyrrolidones, polyvinyl alcohols, anionic (meth)acrylate copolymers of methyl methacrylate and/or ethyl acrylate and methacrylic acid (EUDRAGIT® L 100, EUDRAGIT® S 100, EUDRAGIT® L 100-55). Anionic (meth)acrylate copolymers of methyl methacrylate, methyl acrylate and methacrylic acid of the prior art (see, for example, EP-A 0 704 207 or EP-A 0 704 208), carboxymethylcellulose salts, hydroxypropylcellulose (HPMC), neutral (meth)acrylate copolymers of methyl methacrylate and ethyl acrylate (dry matter from EUDRAGIT® NE 30 D), copolymers of methyl methacrylate and butyl methacrylate (PLASTOID® B) or (meth)acrylate copolymers having quaternary amino groups (EUDRAGIT® RL and EUDRAGIT® RS).

Problem and Solution

EP 0 152 038 A2 starts from pharmaceutical forms with coatings of carboxyl group-containing polymers. These carboxyl group-containing polymers, especially methacrylic acid-containing (meth)acrylate copolymers, are resistant to gastric juices and at the same time soluble in intestinal juice, however. Depending on the content of carboxyl groups, they dissolve at a specific pH. Pharmaceutical forms coated with a polymer of equal parts of ethyl acrylate and methacrylic acid release the active ingredient rapidly, e.g. from about pH 5.5 onwards. According to EP 0 152 038 A2, the effect observed on admixture of water-insoluble, film-forming polymers is that the dissolution pH is shifted upwards, but the active ingredient release characteristics or the time course thereof remains substantially uninfluenced. The effect of the mixture can be described as pH shift. If it is wished to influence the time course of the active ingredient release characteristics, this is evidently possible only by modifying the monomer composition of the carboxyl group-containing polymer. A person skilled in the art of pharmaceutical technology is confronted by the problem that only a limited number of polymers is available. He would therefore need to develop novel polymers with novel monomer compositions in order to obtain variants with which different time courses of the active ingredient release characteristics can be produced with the same dissolution pH.

It was therefore intended to find a solution which makes it possible to modify in a simple manner the time course of the active ingredient release characteristics of anionic or carboxyl group-containing polymers without at the same time influencing the dissolution pH thereof.

The problem is solved by the

use of a mixture of 2 to 60% by weight of one or more polymers (I) with 40 to 98% by weight of one or more polymers (II), where

polymer (I) is a (meth)acrylate copolymer comprising 90 to 100% by weight free radically polymerized units of 40 to 95% by weight of C₁- to C₄-alkyl esters of acrylic or of methacrylic acid and 5 to 60% by weight units of (meth)acrylate monomers having an anionic group, and 0 to 10% by weight of further vinylically polymerizable monomers, and

polymer (II) is a vinyl polymer different from polymer (I) or a polysaccharide or a derivative of a polysaccharide comprising 88 to 100% neutral monomer units and up to 12% by weight polymerized monomer units having ionic radicals,

for the production of a coated pharmaceutical form comprising an active ingredient-containing core and a polymeric coating of the mixture of polymers (I) and (II)
characterized in that

the glass transition temperature of polymer (I) is not more than 70° C., and an active ingredient release profile in which the active ingredient is released by comparison with a pharmaceutical form coated with polymer (I) alone starting at the same pH but more slowly is attained.

The (meth)acrylate copolymers described in EP 0 152 038 A2, e.g. EUDRAGIT® L or EUDRAGIT® L100-55, have glass transition temperatures above 100° C. Polymers of this type are unsuitable as polymer (I) for the purposes of the invention. The invention is based on the realization that the pH-shift effect described in EP 0 152 038 A2 for the polymer mixtures described therein does not occur on selection of anionic or carboxyl group-containing polymers whose glass transition temperature is not above 70° C. There is found with these polymers, entirely surprisingly, the effect according to the problem of the time course of the active ingredient release characteristics being modified without modifying the dissolution pH.

Some of the comprised polymer mixtures are disclosed in principle in WO 03/072087 and WO 2004/096185. It was to be assumed according to the broad teaching of EP 0 152 038 A2 that the mixtures described therein would lead to a pH-shift effect which is unwanted according to the invention. The use of selected mixtures from WO 03/072087 and WO 2004/096185 for solving the stated problem thus opens up new prospects for pharmaceutical technology. A person skilled in the art is able, starting from active ingredient release characteristics, soluble in intestinal juice, of anionic or carboxyl group-containing polymers with assigned specific dissolution pH values to adjust the time course of the active ingredient release characteristics via the mixing ratio of the polymers. It is possible thereby to avoid elaborate alternative developments, specific complicated coating formulations or the development of polymers with alternative monomer composition.

Copolymers of 10 to 30% by weight methyl methacrylate, 50 to 70% by weight methyl acrylate and 5 to 15% by weight methacrylic acid are disclosed in EP 0 704 208 A2. Mixtures with other polymers corresponding to the type of polymer (II) described herein have evidently not previously been suggested. Once again, according to the broad teaching of EP 0 152 038 A2, it would have been expected that such mixtures would lead to the known pH-shift effect. Once again, the invention is based on the realization that the pH-shift effect described in EP 0 152 038 A2 for the polymer mixtures described therein does not occur on selection of anionic or carboxyl group-containing polymers whose glass transition temperature is not above 70° C.; on the contrary, the result is the effect according to the problem, of modifying the time course of the active ingredient release characteristics without modifying the dissolution pH.

The problem is therefore also solved in particular by a pharmaceutical form comprising an active ingredient-containing core which is coated with a mixed polymeric coating, characterized in that the mixture coating is a mixture of 2 to 60% by weight of a polymer (I) with 40 to 95% by weight and one or more polymers (II),
characterized in that

polymer (I) is a copolymer of 10 to 30% by weight methyl methacrylate, 50 to 70% by weight methyl acrylate and 5 to 15% by weight methacrylic acid, and polymer (II) is a vinyl polymer different from polymer (I) and composed of 90 to 100% neutral vinylically polymerized monomer units and may comprise up to 10% by weight vinylically polymerized monomer units having ionic radicals.

### IMPLEMENTATION OF THE INVENTION

Mixing Ratios of Polymer (I) to Polymer (II)

The mixture comprises or consists substantially or preferably 100% of 2 to 60, preferably 2 to 30, % by weight of one or more polymer (I) and 40 to 98, preferably 70 to 98, % by weight of one or more polymers (II). It is possible in this range to adjust nearly all transitions between the release profiles of polymers (I) and (II), so that a novel alternative for formulating pharmaceutical forms is available to a person skilled in the art.

A preferred mixture comprises or consists substantially or preferably 100% of 2 to 15% by weight of one or more polymers (I) with 85 to 98% by weight of one or more polymers (II). In this range, surprisingly even a relatively small proportion of the polymer (I) diverts the unwantedly strongly delaying release characteristics of polymer (II) into a range which is desirable from the therapeutic viewpoint for a long-lasting, nearly constant release of a large number of active ingredients in the various sections of the intestine. The release of the active ingredient at the pH at which the polymer (I) starts to dissolve, in the USP release test (USP 28-NF23), is preferably less than 50% in 60 minutes. It is in particular beneficial for the release of active ingredient at the pH at which the polymer (I) starts to dissolve, in the USP release test, to be more than 10% in 60 minutes.

In this connection, the degree of release is always also influenced by the layer thickness of the coating. This can be increased or reduced with the preset mixing ratio in order to control the release into the desired range.

The active ingredient release can be determined according to USP, in particular USP 28-NF23, General Chapter <711>, Dissolution, Apparatus 2, (Paddle), Method <724> “Delayed Release (Enteric Coated) Articles-General General Drug Release Standard”, Method B (100 rpm, 37° C.) with the following modification: the coated pellets were initially tested in simulated gastric fluid (USP) at pH 1.2 for resistance to gastric fluid for 120 min, and then the buffer is changed to phosphate buffer of pH 7.5, equivalent to a simulated intestinal environment. The active ingredient concentration in the test medium can be determined for example by photometry, depending on the active ingredient.

Polymers (I)

Glass Transition Temperature

The glass transition temperature of polymer (I) is not more than 70° C., preferably 45 to 68° C.

Glass transition temperature means here in particular the midpoint temperature Tmg as defined in ISO 11357-2, subclause 3.3.3. The measurement takes place without added plasticizer, with residual monomer contents (REMO) of less than 100 ppm, with a heating rate of 10° C./min and under a nitrogen atmosphere.

Composition of Polymer (I)

Polymers (I) are (meth)acrylate copolymers comprising or consisting 90 to 100, preferably 95 to 100, particularly preferably 100, % by weight of 40 to 95, preferably 66 to 95, % by weight free-radically polymerized units of C₁- to C₄-alkyl esters of acrylic or of methacrylic acid and 5 to 60, preferably 5 to 34, % by weight units of (meth)acrylate monomers having an anionic group. It is possible where appropriate for 0 to 10% by weight residues of further vinylically polymerizable monomers to be present in polymer (I).

C₁- to C₄-alkyl esters of acrylic or methacrylic acid are in particular methyl methacrylate, ethyl methacrylate, butyl methacrylate, methyl acrylate, ethyl acrylate and butyl acrylate.

A (meth)acrylate monomer having an anionic group may be for example acrylic acid, but preferably methacrylic acid.

The stated proportions of the C₁- to C₄-alkyl esters of acrylic or of methacrylic acid and of the (meth)acrylate monomers having an anionic group ordinarily add up to 100% by weight. Most commercially available polymers (I) comprise no residues of further monomer types.

However, it is additionally possible, without this leading to an impairment or alteration in the essential properties of polymers (I), for small amounts in the range from 0 to 10, e.g. 1 to 5, % by weight of further vinylically copolymerizable monomers such as, for example, hydroxyethyl methacrylate or hydroxyethyl acrylate, butyl acrylate, vinylpyrrolidone, vinylmalonic acid, styrene, vinyl alcohol, vinyl acetate and/or derivatives thereof to be present. However, it is preferred for no further vinylically copolymerizable monomers to be present.

The glass transition temperature of polymer (I) is not more than 70, preferably 40 to 70, particularly preferably 45 to 65, in particular 45 to 55° C.

Glass transition temperature means here in particular the midpoint temperature Tmg as defined in ISO 11357-2, subclause 3.3.3. The measurement takes place without added plasticizer, with residual monomer contents (REMO) of less than 100 ppm, with a heating rate of 10° C./min and under a nitrogen atmosphere.

Dispersions/Partial Neutralization

The polymer (I) is ordinarily an emulsion polymer and is preferably produced and used in the form of a 10 to 50 percent by weight, in particular 20 to 40 percent, aqueous dispersion. A solids content of 30% by weight is preferred as commercial form. Partial neutralization of the methacrylic acid units can be dispensed with for processing; however, it is possible, for example to an extent of up to 5 or 10 mol %, should a stabilization or thickening of the coating agent dispersion be desired. The weight average latex particle size (radius) is ordinarily 40 to 100 nm, preferably 50 to 70 nm, thus ensuring a viscosity below 1000 mPa·s which is beneficial for processing technology. The particle size can be determined by laser diffraction, e.g. using a Mastersizer 2000 (from Malvern).

With a higher degree of neutralization, e.g. 10 to 50 mol %, or complete neutralization, it is possible to convert the copolymer into a dissolved state.

To prepare a solution of the anionic copolymer it is ordinarily necessary for the acidic groups to be partially or completely neutralized. The anionic copolymer can for example be gradually stirred into water in a final concentration of 1 to 40% by weight and, at the same time, be partially or completely neutralized by adding a basic substance such as, for example, NaOH, KOH, ammonium hydroxide or organic bases such as, for example, triethanolamine. It is also possible to employ a powder of the copolymer to which a base, e.g. NaOH, has been added during its preparation for the purpose of (partial) neutralization, so that the powder is an already (partially) neutralized polymer. The pH of the solution is ordinarily above 4, e.g. in the range from 4 to about 7. It is moreover possible also for batches of completely or partially neutralized dispersions to be mixed with unneutralized dispersions and further processed in the manner described, i.e. the mixture can be used for coatings or be initially freeze dried or spray dried to give a powder.

The dispersion can also for example be spray dried or freeze dried in a manner known per se and be provided in the form of a redispersible powder (see, for example, EP-A 0 262 326). Alternative processes are freeze drying or coagulation and squeezing out the water in an extruder with subsequent granulation (see, for example, EP-A 0 683 028).

It has surprisingly been found that copolymer dispersions of spray-dried or freeze-dried and redispersed powders exhibit increased shear stability. This is advantageous in particular for spray application. This advantage is strongly evident in particular when the copolymer present in the dispersion is partially neutralized to the extent of 2 to 10, preferably 5 to 7, mol % (based on the acidic groups present in the copolymer). It is preferred to add NaOH for the partial neutralization for this purpose. An anionic emulsifier is preferably present in an amount of 0.1 to 2% by weight. Sodium lauryl sulfate is particularly preferred as emulsifier.

Polymer (I) Type with 5 to 15% by Weight Methacrylic Acid

Suitable polymers (I), disclosed in EP 0 704 208 A2, are (meth)acrylate polymers consisting of 10 to 30% by weight methyl methacrylate, 50 to 70% by weight methyl acrylate and 5 to 15% by weight methacrylic acid (EUDRAGIT® FS type). The pH at the start of the specific release of active ingredient in intestinal juice or simulated intestinal fluid can be stated to be pH 7.0. The glass transition temperature of this polymer (I) is preferably 45 to 55° C.

EUDRAGIT® FS is a copolymer of 25% by weight methyl methacrylate, 65% by weight methyl acrylate and 10% by weight methacrylic acid. EUDRAGIT® FS 30 D is a dispersion comprising 30% by weight EUDRAGIT® FS. The glass transition temperature T_{mg} according to ISO 11357-2, subclause 3.3.3, is about 48° C.

Polymer (I) Type with 20 to 34% by Weight Methacrylic Acid and Ultimate Elongation Properties

Further suitable polymers (I) are copolymers disclosed in WO 03/072087, of
20 to 34% by weight methacrylic acid and/or acrylic acid,
20 to 69% by weight methyl acrylate and
0 to 40% by weight ethyl acrylate and/or optionally
0 to 10% by weight further vinylically copolymerizable monomers,
   with the proviso that the proportions of monomers are chosen so that the glass transition temperature of the copolymer according to ISO 11357-2, subclause 3.3.3, is not more than 60° C. This (meth)acrylate copolymer is particularly suitable, because of its good ultimate elongation properties, for compression of pellets to give tablets.

The abovementioned copolymer is composed in particular of free radically polymerized units of

20 to 34, preferably 25 to 33, particularly preferably 28 to 32, % by weight methacrylic acid or acrylic acid, with preference for methacrylic acid,

20 to 69, preferably 35 to 65, particularly preferably 35 to 55, % by weight methyl acrylate and optionally

0 to 40, preferably 5 to 35, particularly preferably 15 to 35, % by weight ethyl acrylate, with the proviso that the glass transition temperature of the copolymer (measurement without added plasticizer with a residual monomer content (REMO) of less than 100 ppm, heating rate 10° C./min, nitrogen atmosphere) according to ISO 11357-2, subclause 3.3.3 (T_{mg}), is not more than 60, preferably 40 to 60, particularly preferably 45 to 55° C.

The copolymer preferably consists substantially to exclusively of the monomers methacrylic acid, methyl acrylate and ethyl acrylate in the quantitative proportions indicated above.

However, it is additionally possible, without this leading to an impairment of the essential properties, for small amounts in the range from 0 to 10, e.g. 1 to 5, % by weight of further vinylically copolymerizable monomers such as, for example, methyl methacrylate, butyl methacrylate, butyl acrylate or hydroxyethyl methacrylate, to be present.

It is also possible to employ mixtures of the said copolymers to adjust specific release profiles or release sites.

Glass transition temperature means here in particular the midpoint temperature Tmg as defined in ISO 11357-2, subclause 3.3.3. The measurement takes place without added plasticizer, with residual monomer contents (REMO) of less than 100 ppm, with a heating rate of 10° C./min and under a nitrogen atmosphere.

The copolymers are obtained in a manner known per se by free-radical bulk, solution, bead or emulsion polymerization. They must be brought before processing into the particle size range according to the invention by suitable grinding, drying or spraying processes. This can take place by simple crushing of extruded and cooled pellets or hot cut.

The use of powders may be advantageous, especially in the case of mixing with further powders or liquids. Suitable items of apparatus for producing the powders are familiar to the person skilled in the art, e.g. air jet mills, pinned disc mills, compartment mills. It is possible where appropriate to include appropriate sieving steps. A suitable mill for industrial large quantities is for example an opposed jet mill (Multi No. 4200) which is operated with a gage pressure of about 6 bar.

Polymer (I) Type with 20 to 33% by Weight Methacrylic Acid with Good Mechanical Properties, in Particular for Compression of Pellets to Give Tablets.

Further suitable polymers (I) are copolymers disclosed in WO 2004/096185, of
20 to 33% by weight methacrylic acid and/or acrylic acid,
5 to 30% by weight methyl acrylate and
20 to 40% by weight ethyl acrylate and more than 10 to 30% by weight butyl methacrylate and optionally
0 to 10% by weight further vinylically copolymerizable monomers, where the proportions of the monomers add up to 100% by weight,
   with the proviso that the proportions of monomers are chosen so that the glass transition temperature of the copolymer according to ISO 11357-2, subclause 3.3.3 (midpoint temperature T_{mg}), is 55 to 70° C. Copolymers of this type are particularly suitable, because of their good mechanical properties, for compressing pellets to give tablets.

The abovementioned copolymer is composed in particular of free radically polymerized units of

20 to 33, preferably 25 to 32, particularly preferably 28 to 31, % by weight methacrylic acid or acrylic acid, with preference for methacrylic acid,

5 to 30, preferably 10 to 28, particularly preferably 15 to 25, % by weight methyl acrylate,

20 to 40, preferably 25 to 35, particularly preferably 18 to 22, % by weight ethyl acrylate, and

more than 10 to 30, preferably 15 to 25, particularly preferably 18 to 22, % by weight butyl methacrylate,

where the monomer composition is chosen so that the glass transition temperature of the copolymer is 55 to 70° C., preferably 59 to 66, particularly preferably 60 to 65° C.

The copolymer preferably consists substantially to exclusively, to the extent of 90, 95 or 99 to 100% by weight, of the monomers methacrylic acid, methyl acrylate, ethyl acrylate and butyl methacrylate in the quantitative ranges indicated above.

However, it is additionally possible, without this necessarily leading to an impairment of the essential properties, for small amounts in the range from 0 to 10, e.g. 1 to 5, % by weight of further vinylically copolymerizable monomers such as, for example, methyl methacrylate, butyl acrylate, hydroxyethyl methacrylate, vinylpyrrolidone, vinylmalonic acid, styrene, vinyl alcohol, vinyl acetate and/or derivatives thereof to be present.

Polymers (II)

Polymer (II) is a vinyl polymer different from polymer (I), or a polysaccharide or a derivative of a polysaccharide which is composed to the extent of 80 to 100% of neutral monomer units and may comprise up to 12% by weight monomer units having ionic radicals.

Vinyl Polymers

Polymer (II) may be a vinyl polymer comprising 88 to 100% neutral vinylically polymerized monomer units and up to 12% by weight vinylically polymerized monomer units having ionic radicals.

Polymer (II) may be a copolymer of methyl methacrylate and ethyl acrylate, a copolymer of methyl methacrylate and ethyl acrylate and methacrylic acid, a copolymer of methyl methacrylate, ethyl acrylate and trimethylammoniumethyl methacrylate, a polyvinylpyrrolidones (PVP), polyvinyl alcohols, polyvinyl alcoholpolyethylene glycol graft copolymer (KollicoatO IR), polyvinyl acetate (PVAc, Kollicoat® SR), vinyl acetatevinylpyrrolidone copolymer (Kollidone® VA64), vinyl acetate: crotonic acid 9:1 copolymer (VAC:CRA, Kollicoat® VAC),

Polysaccharides or Derivatives

Polymer (II) may be a polysaccharide or the derivative of a polysaccharide comprising 88 to 100% neutral monomer units and up to 12W by weight polymerized monomer units having ionic radicals.

Polymer (II) may be: starch and derivatives thereof, hydroxyethylcellulose (HEC, Klucel®, hydroxypropylcellulose (HPC), hydroxypropylmethylcellulose (HPMC, Pharmacoat®, Methocel®, Sepifilm®, Viscontran®, Opadry®), hydroxymethylethylcellulose (HEMC), ethylcellulose (EC, Ethocel®, Aquacoat®, Surelease®), methylcellulose (MC, Viscontran®, Tylopur®, Methocel®), cellulose esters, cellulose glycolate or a mixture of said polymers.

(Meth)acrylate Copolymers

Neutral (meth)acrylate Copolymers

Polymer (II) may be in particular a (meth)acrylate copolymer which is different from polymer (I) and comprises 88 to 100% neutral monomer units and up to 12% by weight polymerized monomer units having ionic radicals.

Neutral methyl acrylate copolymers which have been prepared in accordance with WO 01/68767 as dispersions using 1-10% by weight of a nonionic emulsifier with an HLB of 15.2 to 17.3 are preferred. The latter have the advantage that a phase separation with formation of crystal structures is suppressed by the emulsifier.

Polymer (II) may particularly preferably be a copolymer of 20 to 40% by weight ethyl acrylate and 60 to 80% by weight methyl methacrylate (EUDRAGIT® NE type).

Particularly suitable as polymer (II) is a copolymer of 30% by weight ethyl acrylate and 70% by weight methyl methacrylate (EUDRAGIT® NE).

(Meth)acrylate Copolymers having Quaternary Amino Groups

Polymer (II) may furthermore be composed of 88 to 98% by weight free radically polymerized C₁- to C₄-alkyl esters of acrylic or of methacrylic acid and 12 to 2% by weight (meth)acrylate monomers having a quaternary amino group in the alkyl radical.

Preferred C₁- to C₄-alkyl esters of acrylic or of methacrylic acid are methyl acrylate, ethyl acrylate, butyl acrylate, butyl methacrylate and methyl methacrylate.

The particularly preferred (meth)acrylate monomer having quaternary amino groups is 2-trimethylammoniummethyl methacrylate chloride.

Polymer (II) may be a copolymer of 50-70% by weight methyl methacrylate, 20-40% by weight ethyl acrylate and 12-2 by weight trimethylammoniumethyl methacrylate chloride (EUDRAGIT® RS/RL type).

A specifically suitable copolymer comprises 65% by weight methyl methacrylate, 30% by weight ethyl acrylate and 5% by weight 2-trimethylammoniummethyl methacrylate chloride be composed (EUDRAGIT® RS). A specifically suitable copolymer comprises 60% by weight methyl methacrylate, 30% by weight ethyl acrylate and 10% by weight 2-trimethylammoniummethyl methacrylate chloride be composed (EUDRAGIT® RL).

Active ingredient-containing pellets can be produced by applying active ingredient by means of a layering process. For this purpose, active ingredient is homogenized together with further excipients (mold release agents, where appropriate plasticizer) and dissolved or suspended in a binder. The liquid can be applied by means of a fluidized bed process to placebo pellets or other suitable carrier materials, with evaporation of the solvent or suspending agent (literature: International Journal of Pharmaceutics 143, pp. 13-23). The production process may be followed by a drying step. The active ingredient can be applied in a plurality of layers.

Some active ingredients, e.g. acetylsalicylic acid, are commercially available in the form of active ingredient crystals and can be employed in this form instead of active ingredient-containing pellets.

Film coatings on active ingredient-containing pellets are normally applied in fluidized bed apparatuses. Formulation examples are mentioned in this application. Film formers are normally mixed with plasticizers and mold release agents by a suitable process. It is possible in this case for the film formers to be in the form of a solution or suspension. The excipients for the film formation may likewise be dissolved or suspended. organic or aqueous solvents or dispersants can be used. It is additionally possible to use stabilizers to stabilize the dispersion (example: Tween 80 or other suitable emulsifiers or stabilizers).

Examples of mold release agents are glycerol mono-stearate or other suitable fatty acid derivatives, silica derivatives or talc. Examples of plasticizers are propylene glycol, phthalates, polyethylene glycols, sebacates or citrates, and other substances mentioned in the literature.

It is possible to apply between active ingredient-containing layer and copolymer layer according to the invention a separating layer which serves to separate active ingredient and coating material for the purpose of preventing interactions. This layer may consist of inert film formers (e.g. HPMC, HPC or (meth)acrylic acid copolymers) or, for example, talc or other suitable pharmaceutical substances. It is likewise possible to use combinations of film formers and talc or similar substances.

It is also possible to apply a separating layer composed of partially or completely neutralized copolymer dispersions.

Mixtures for producing tablets from coated particles are prepared by mixing the pellets with suitable binders for tableting, if necessary adding disintegration-promoting substances, and if necessary adding lubricants. The mixing can take place in suitable machines. Unsuitable mixers are those leading to damage to the coated particles, e.g. plowshare mixers. A specific sequence of addition of the excipients to the coated particles may be necessary to achieve suitable short disintegration times. It is possible by premixing with the coated particles with the lubricant or mold release agent magnesium stearate to render its surface hydrophobic and thus prevent adhesion.

Mixtures suitable for tableting normally comprise 3 to 15% by weight of a disintegration aid, e.g. Kollidon CL and, for example, 0.1 to 1% by weight of a lubricant and mold release agent such as magnesium stearate. The proportion of binder is determined according to the required proportion of coated particles.

Examples of typical binders are Cellactose®, microcrystalline cellulose, calcium phosphates, Ludipress®, lactose or other suitable sugars, calcium sulfates or starch derivatives. Substances of low bulk density are preferred.

Typical disintegration aids (disintegrants) are crosslinked starch or cellulose derivatives, and crosslinked polyvinylpyrrolidone. Cellulose derivatives are likewise suitable. The use of disintegration aids can be dispensed with through selection of a suitable binder.

Typical lubricants and mold release agents are magnesium stearates or other suitable salts of fatty acids or substances mentioned in the literature for this purpose (e.g. lauric acid, calcium stearate, talc etc.). The use of a lubricant and mold release agent in the mixture can be dispensed with on use of suitable machines (e.g. tablet press with external lubrication) or suitable formulations.

A flow-improving aid can be added where appropriate to the mixture (e.g. colloidal silica derivatives, talc etc.).

The tableting can take place on conventional tablet presses, eccentric or rotary tablet presses, with compressive forces in the range from 5 to 40 kN, preferably 10-20 kN. The tablet presses may be equipped with systems for external lubrication. Special systems for die filling which avoid die filling by means of impeller paddles are employed where appropriate.

Polymer Mixture

Firstly, a mixture of one or more polymer (I) and one or more polymer (II) is prepared. For this purpose, for example, two organic solutions or two aqueous dispersions are mixed in proportion. Preferably, the mixture of aqueous dispersions of one or more of polymer (I) and one or more of polymer (II) is prepared. Ordinarily, in each case one polymer (I) and one polymer (II) will be employed. The mixture comprises 2 to 60, preferably 10 to 55, % by weight of a polymer (I) with 40 to 98, preferably 45 to 90, % by weight of one or more polymers (II), with the proportions amounting to 100% by weight. Ordinarily, but not obligatorily, pharmaceutically usual excipients are additionally admixed, which are dissolved or dispersed separately where appropriate.

Pharmaceutical Form

The invention further relates to a pharmaceutical form with a selected polymer (I) which is not evident from EP 0 152 038 A2. The polymer (II) present in the pharmaceutical form is identical to the polymers (II) described herein and employed according to the use.

The invention accordingly relates to a pharmaceutical form comprising an active ingredient-containing core which is coated with a mixed polymeric coating, characterized in that the mixed coating is a mixture of 2 to 60% by weight of a polymer (I) with 40 to 95% by weight and one or more polymers (II),
characterized in that

polymer (I) is a copolymer of 10 to 30% by weight methyl methacrylate, 50 to 70% by weight methyl acrylate and 5 to 15% by weight methacrylic acid, and

polymer (II) is a vinyl polymer different from polymer (I), or a polysaccharide or a derivative of a polysaccharide which is composed to the extent of 88 to 100% of neutral monomer units and may comprise up to 12% by weight monomer units having ionic radicals.

Polymer (I) Type with 5 to 15% by Weight Methacrylic Acid

Suitable polymers (I) for the pharmaceutical form of the invention are disclosed in EP 0 704 208 A2. Polymers (I) are (meth)acrylate copolymers consisting of 10 to 30% by weight methyl methacrylate, 50 to 70% by weight methyl acrylate and 5 to 15% by weight methacrylic acid (EUDRAGIT® FS type). The pH at the start of the specific release of active ingredient in intestinal juice or simulated intestinal fluid can be stated to be pH 7.0. The glass transition temperature of this polymer (I) is preferably 45 to 55° C.

EUDRAGIT® FS is a copolymer of 25% by weight methyl methacrylate, 65% by weight methyl acrylate and 10% by weight methacrylic acid. EUDRAGIT® FS 30 D is a dispersion comprising 30% by weight EUDRAGIT® FS. The glass transition temperature T_{mg} according to ISO 11357-2, subclause 3.3.3, is about 48° C.

General Process for Producing the Described Pharmaceutical Forms

Cores

Carriers for the coatings are capsules, tablets, granules, pellets, crystals of regular or irregular shape. The size of granules, pellets or crystals is between 0.01 and 2.5 mm, and that of tablets is between 2.5 and 30.00 mm. Capsules consist of gelatin, starch or cellulose derivatives.

They ordinarily comprise the bioactive substance (active ingredient) to the extent of up to 95%, and further pharmaceutical excipients to the extent of up to 99.9% by weight. Conventional processes for production are direct compression, compression of dry, wet or sintered granules, extrusion and subsequent rounding, wet or dry granulation or direct pelleting (e.g. on plates) or by binding powders (powder layering) onto active ingredient-free beads (nonpareilles) or active ingredient-containing particles.

Besides the active ingredient, they may comprise further pharmaceutical excipients: binders such as cellulose and derivatives thereof, polyvinylpyrrolidone (PVP), humectants, disintegration promoters, lubricants, disintegrants, (meth)acrylates, starch and derivatives thereof, sugar solubilizers or others.

Production of a Pharmaceutical Form

Active ingredient-containing pellets can be produced by applying active ingredient by means of a layering process. For this purpose, active ingredient is homogenized together with further excipients (mold release agents, where appropriate plasticizer) and dissolved or suspended in a binder. The liquid can be applied by means of a fluidized bed process to placebo pellets or other suitable carrier materials, with evaporation of the solvent or suspending agent (literature: International Journal of Pharmaceutics 143, pp. 13-23). The production process may be followed by a drying step. The active ingredient can be applied in a plurality of layers.

Some active ingredients, e.g. acetylsalicylic acid, are commercially available in the form of active ingredient crystals and can be employed in this form instead of active ingredient-containing pellets.

Firstly, a mixture of polymer (I) and of polymer (II) is prepared. For this purpose, for example, two dispersions are mixed in proportion.

Film coatings on active ingredient-containing pellets are normally applied in fluidized bed apparatuses. Formulation examples are mentioned in this application. Film formers are normally mixed with plasticizers and mold release agents by a suitable process. It is possible in this case for the film formers to be in the form of a solution or suspension. The excipients for the film formation may likewise be dissolved or suspended. Organic or aqueous solvents or dispersants can be used. It is additionally possible to use stabilizers to stabilize the dispersion (example: Tween 80 or other suitable emulsifiers or stabilizers).

Examples of mold release agents are glycerol mono-stearate or other suitable fatty acid derivatives, silica derivatives or talc. Examples of plasticizers are propylene glycol, phthalates, polyethylene glycols, sebacates or citrates, and other substances mentioned in the literature.

It is possible to apply between active ingredient-containing layer and coating layer according to the invention a separating layer which serves to separate active ingredient and coating material for the purpose of preventing interactions. This layer may consist of inert film formers (e.g. HPMC, HPC or (meth)acrylic acid copolymers) or, for example, talc or other suitable pharmaceutical substances. It is likewise possible to use combinations of film formers and talc or similar substances.

It is also possible to apply a separating layer composed of partially or completely neutralized copolymer dispersions.

Polymer Coating

The polymer coating may preferably for example amount to 2 to 20% by weight in relation to the weight of the active ingredient-containing core. The degree of release is moreover always also influenced by the layer thickness of the coating. This can be increased or reduced with the preset mixing ratio in order to control the release into the desired range.

Topcoat

It is also possible to apply an outer covering layer (topcoat) of a further, preferably water-soluble, polymer and excipients, e.g. pigments and/or mold release agents, which ensures further functions such as, for example, coloring or prevention of adhesion.

Production of Multiparticulate Pharmaceutical Forms

The coated pharmaceutical form is preferably in the form of pellets which are present in a multiparticulate pharmaceutical form, in particular in pellet-containing tablets, minitablets, capsules, sachets or reconstitutable powders.

The invention is particularly suitable for the production of multiparticulate pharmaceutical forms because the mixture according to the invention withstands the high pressures during compression of the pellets with the filler. The coated pharmaceutical form is preferably in the form of pellets which are present in a multiparticulate pharmaceutical form, in particular in pellet-containing tablets, minitablets, capsules, sachets or reconstitutable powders.

The production of multiparticulate pharmaceutical forms by compression of a pharmaceutically usual binder with active ingredient-containing particles is described in detail for example Beckert et al. (1996), “Compression of enteric-coated pellets to disintegrating tablets”, International Journal of Pharmaceutics 143, pp. 13-23 and in WO 96//1624.

Active ingredient-containing pellets can be produced by applying active ingredient by means of a layering process. For this purpose, active ingredient is homogenized together with further excipients (mold release agents, where appropriate plasticizer) and dissolved or suspended in a binder. The liquid can be applied by means of a fluidized bed process to placebo pellets or other suitable carrier materials, with evaporation of the solvent or suspending agent (literature: International Journal of Pharmaceutics 143, pp. 13-23). The production process may be followed by a drying step. The active ingredient can be applied in a plurality of layers.

Some active ingredients, e.g. acetylsalicylic acid, are commercially available in the form of active ingredient crystals and can be employed in this form instead of active ingredient-containing pellets.

Film coatings on active ingredient-containing pellets are normally applied in fluidized bed apparatuses. Formulation examples are mentioned in this application. Film formers are normally mixed with plasticizers and mold release agents by a suitable process. It is possible in this case for the film formers to be in the form of a solution or suspension. The excipients for the film formation may likewise be dissolved or suspended. Organic or aqueous solvents or dispersants can be used. It is additionally possible to use stabilizers to stabilize the dispersion (example: Tween 80 or other suitable emulsifiers or stabilizers).

Examples of mold release agents are glycerol monostearate or other suitable fatty acid derivatives, silica derivatives or talc. Examples of plasticizers are propylene glycol, phthalates, polyethylene glycols, sebacates or citrates, and other substances mentioned in the literature.

Mixtures for producing tablets from coated particles are prepared by mixing the pellets with suitable binders for tableting, if necessary adding disintegration-promoting substances, and if necessary adding lubricants. The mixing can take place in suitable machines. Unsuitable mixers are those leading to damage to the coated particles, e.g. plowshare mixers. A specific sequence of addition of the excipients to the coated particles may be necessary to achieve suitable short disintegration times. It is possible by premixing with the coated particles with the lubricant or mold release agent magnesium stearate to render its surface hydrophobic and thus prevent adhesion.

Mixtures suitable for tableting normally comprise 3 to 15% by weight of a disintegration aid, e.g. Kollidon CL and, for example, 0.1 to 1% by weight of a lubricant and mold release agent such as magnesium stearate. The proportion of binder is determined according to the required proportion of coated particles.

Examples of typical binders are Cellactose®, microcrystalline cellulose, calcium phosphates, Ludipress®, lactose or other suitable sugars, calcium sulfates or starch derivatives. Substances of low bulk density are preferred.

Typical disintegration aids (disintegrants) are crosslinked starch or cellulose derivatives, and cross-linked polyvinylpyrrolidone. Cellulose derivatives are likewise suitable. The use of disintegration aids can be dispensed with through selection of a suitable binder.

Typical lubricants and mold release agents are magnesium stearates or other suitable salts of fatty acids or substances mentioned in the literature for this purpose (e.g. lauric acid, calcium stearate, talc etc.). The use of a lubricant and mold release agent in the mixture can be dispensed with on use of suitable machines (e.g. tablet press with external lubrication) or suitable formulations.

A flow-improving aid can be added where appropriate to the mixture (e.g. colloidal silica derivatives, talc etc.).

The tableting can take place on conventional tablet presses, eccentric or rotary tablet presses, with compressive forces in the range from 5 to 40 kN, preferably 10-20 kN. The tablet presses may be equipped with systems for external lubrication. Special systems for die filling which avoid die filling by means of impeller paddles are employed where appropriate.

Active Ingredient Release

The active ingredient release profile obtained according to the invention is one in which the active ingredient is released by comparison with a pharmaceutical form coated with polymer (I) alone starting at the same pH but more slowly.

The active ingredient release profile obtained according to the invention is one in which the active ingredient is released by comparison with a pharmaceutical form coated with polymer (II) alone starting at the same pH but more rapidly.

Preferred pharmaceutical forms are those in which the release of active ingredient at a pH at which polymer (I) starts to dissolve is, in the USP release test (USP 28-NF23), less than 50%, preferably less than 25%, particularly preferably 10 to 50%, in 60 minutes. The release test, e.g. according to USP (according to USP 28-NF23, method B, modified test for enteric coated products) is known to the person skilled in the art. The test conditions are in particular: paddle method, 100 revolutions per minute, 37° C.; pH 1.2 with 0.1 N HCl, pH 7.5 by addition of 0.2 M phosphate buffer and adjustment with 2 N NaOH. See also USP 27-NF22 supplement 1, delayed release method, monograph <724> drug release.

Excipients Customary in Pharmacy

Excipients customary in pharmacy are added to the formulation of the invention, preferably during production of the granules or powders. The additives can also be added to the coating agent and binder during processing. It is, of course, always necessary for all the substances employed to be toxicologically acceptable and usable in particular in medicaments without a risk for patients.

The amounts employed and the use of the usual additives in medicament coatings or layerings are familiar to the person skilled in the art. Examples of possible customary additives are mold release agents, pigments, stabilizers, antioxidants, pore formers, penetration promoters, gloss agents, aromatizing substances or flavorings. They serve as processing aids and are intended to ensure a reliable and reproducible production process and good long-term storage stability, or they achieve additional advantageous properties in the pharmaceutical form. They are added to the polymer preparations before processing and may influence the permeability of the coatings, it being possible to utilize this where appropriate as additional control parameter.

Mold Release Agents:

Mold release agents ordinarily have lipophilic properties and are ordinarily added to the spray suspensions. They prevent agglomeration of the cores during the film coating. Talc, Mg stearate or Ca stearate, ground silica, kaolin or nonionic emulsifiers having an HLB of between 3 and 8 are preferably employed. The usual amounts employed of mold release agents in the coating agents and binders according to the invention are between 0.5 to 100% by weight based on the dry weight of the dispersion.

Pigments:

Pigments incompatible with the coating agent are in particular those pigments which, if added directly to the (meth)acrylate copolymer dispersion, e.g. by stirring in, in the usual amounts used of, for example, 20 to 400% by weight based on the dry weight of the (meth)acrylate copolymer, lead to destabilization of the dispersion, coagulation, to signs of inhomogeneity or similarly unwanted effects. The pigments to be used are moreover of course non-toxic and suitable for pharmaceutical purposes. Concerning this, see also, for example: Deutsche Forschungsgemeinschaft, Farbstoffe fur Lebensmittel, Harald, Boldt Verlag K G, Boppard (1978); Deutsche Lebensmittelrundschau 74, No. 4, p. 156 (1978); Arzneimittelfarbstoffverordnung AmFarbV of 25.08.1980.

Pigments incompatible with the coating agent may be for example alumina pigments. Examples of incompatible pigments are orange yellow, cochineal red lake, colored pigments based on alumina or azo dyes, sulfonic acid dyes, orange yellow S (E110, C.I. 15985, FD&C Yellow 6), indigo carmine (E132, C.I. 73015, FD&C Blue 2), tartrazine (E 102, C.I. 19140, FD&C Yellow 5), Ponceau 4R (E 125, C.I. 16255, FD&C Cochineal Red A), quinoline yellow (E 104, C.I. 47005, FD&C Yellow 10), erythrosine (E127, C.I. 45430, FD&C Red 3), azorubine (E 122, C.I. 14720, FD&C Carmoisine), amaranth (E 123, C.I. 16185, FD&C Red 2), acid brilliant green (E 142, C.I. 44090, FD&C Green S).

The E numbers indicated for the pigments relate to an EU numbering. Concerning this, see also “Deutsche Forschungsgemeinschaft, Farbstoffe fur Lebensmittel, Harald Boldt Verlag K G, Boppard (1978); Deutsche Lebensmittelrundschau 74, No. 4, p. 156 (1978); Arzneimittelfarbstoffverordnung AmFarbV of 25.08.1980. The FD&C numbers relate to the approval in food, drugs and cosmetics by the U.S. food and drug administration (FDA) described in: U.S. Food and Drug Administration, Center for Food Safety and Applied Nutrition, Office of Cosmetics and Colors: Code of Federal Regulations—Title 21 Color Additive Regulations Part 82, Listing of Certified Provisionally Listed Colors and Specifications (CFR 21 Part 82).

Plasticizers

Further additives may also be plasticizers. The usual amounts are between 0 and 50, preferably 5 to 20, % by weight.

Plasticizers may influence the functionality of the polymer layer, depending on the type (lipophilic or hydrophilic) and added amount. Plasticizers achieve through physical interaction with the polymers a reduction in the glass transition temperature and promote film formation, depending on the added amount. Suitable substances usually have a molecular weight of between 100 and 20 000 and comprise one or more hydrophilic groups in the molecule, e.g. hydroxyl, ester or amino groups.

Examples of suitable plasticizers are alkyl citrates, glycerol esters, alkyl phthalates, alkyl sebacates, sucrose esters, sorbitan esters, diethyl sebacate, dibutyl sebacate and polyethylene glycols 200 to 12 000. Preferred plasticizers are triethyl citrate (TEC), acetyl triethyl citrate (ATEC) and dibutyl sebacate (DBS). Mention should additionally be made of esters which are usually liquid at room temperature, such as citrates, phthalates, sebacates or castor oil. Esters of citric acid and sebacic acid are preferably used.

Addition of the plasticizers to the formulation can be carried out in a known manner, directly, in aqueous solution or after thermal pretreatment of the mixture. It is also possible to employ mixtures of plasticizers.

Active Ingredients

Medicinal substances in use can be found in reference works such as, for example, the Rote Liste or the Merck Index.

Biologically Active Substances:

The medicinal substances employed for the purposes of the invention are intended to be used on or in the human or animal body in order
1. to cure, to alleviate, to prevent or to diagnose disorders, conditions, physical damage or pathological symptoms.
2. to reveal the condition, the status or the functions of the body or mental states.
3. to replace active substances or body fluids produced by the human or animal body.
4. to ward off, to eliminate or to render harmless pathogens, parasites or exogenous substances, or
5. to influence the condition, the status or the functions of the body or mental states.

The formulation of the invention is suitable for administration of in principle any active pharmaceutical ingredients or biologically active substances.

Therapeutic Classes

These pharmaceutically active substances may belong to one or more active ingredient classes such as ACE inhibitors, adrenergics, adrenocorticosteroids, acne therapeutic agents, aldose reductase inhibitors, aldosterone antagonists, alpha-glucosidase inhibitors, alpha 1 antagonists, remedies for alcohol abuse, amino acids, amoebicides, anabolics, analeptics, anesthetic additions, anesthetics (non-inhalational), anesthetics (local), analgesics, androgens, angina therapeutic agents, antagonists, antiallergics, antiallergics such as PDE inhibitors, antiallergics for asthma treatment, further antiallergics (e.g. leucotriene antagonists, antianemics, antiandrogens, antianxiolytics, antiarthritics, antiarrhythmics, antiatheriosclerotics, antibiotics, anticholinergics, anticonvulsants, antidepressants, antidiabetics, antidiarrheals, antidiuretics, antidotes, antiemetics, antiepileptics, antifibrinolytics, antiepileptics, antihelmintics, antihistamines, antihypotensives, antihypertensives, antihypertensives, antihypotensives, anticoagulants, antimycotics, antiestrogens, antiestrogens (non-steroidal), antiparkinson agents, antiinflammatory agents, antiproliferative active ingredients, antiprotozoal active ingredients, antirheumatics, antischistosomicides, antispasmolytics, antithrombotics, antitussives, appetite suppressants, arteriosclerosis remedies, bacteriostatics, beta-blockers, beta-receptor blockers, bronchodilators, carbonic anhydrase inhibitors, chemotherapeutic agents, choleretics, cholinergics, cholinergic agonists, cholinesterase inhibitors, agents for the treatment of ulcerative colitis, cyclooxygenaze inhibitors, diuretics, ectoparasiticides, emetics, enzymes, enzyme inhibitors, enzyme inhibitors, active ingredients to counter vomiting, fibrinolytics, fungistatics, gabapentin gout remedies, glaucoma therapeutic agents, glucocorticoids, glucocorticosteroids, hemostatics, cardiac glycosides, histamine H2 antagonists, hormones and their inhibitors, immunotherapeutic agents, cardiotonics, coccidiostats, laxatives, lipid-lowering agents, gastrointestinal therapeutic agents, malaria therapeutic agents, migraine remedies, microbiocides, Crohn's disease, metastasis inhibitors, migraine remedies, mineral preparations, motility-increasing active ingredients, muscle relaxants, neuroleptics, active ingredients for treatment of estrogens, osteoporosis, otologicals, antiparkinson agents, phyto-pharmaceuticals, pitavastatin, proton pump inhibitors, prostaglandins, active ingredients for treating benign prostate hyperblasia, active ingredients for treating pruritus, psoriasis active ingredients, psychoactive drugs, free-radical scavengers, renin antagonists, thyroid therapeutic agents, active ingredients for treating seborrhea, active ingredients to counter seasickness, spasmolytics, alpha- and beta-sympathomimetics, tenatoprazole, platelet aggregation inhibitors, tyrosine kinase inhibitors, tranquilizers, ulcer therapeutic agents, further ulcer therapeutic agents, agents for the treatment of urolithiasis, virustatics, virustatics, vitamins, cytokines, active ingredients for combination therapy with cytostatics, cytostatics.

Active Ingredients

Examples of suitable active ingredients are acarbose, acetylsalicylic acid, abacavir, aceclofenac, aclarubicin, acyclovir, actinomycin, adalimumab, adefovir, adefovirdipivoxil, adenosylmethionine, adrenaline and adrenaline derivatives, agalsidase alpha, agalsidase beta, alemtuzumab, almotriptan, alphacept, allopurinol, almotriptan, alosetron, alprostadil, amantadine, ambroxol, amisulpride, amlodipine, amoxicillin, 5-aminosalicylic acid, amitriptyline, amlodipine, amoxicillin, amprenavir, anagrelide, anakinra, anastrozole, androgen and androgen derivatives, apomorphine, aripiprazole, arsenic trioxide, artemether, atenolol, atorvastatin, atosiban, azathioprine, azelaic acid, barbituric acid derivatives, balsalazide, basiliximab, beclapermin, beclomethasone, bemiparin, benzodiazepines, betahistine, bexaroten, bezafibrate, bicalutamide, bimatoprost, bosentan, botulinus toxim, brimonidine, brinzolamide, budesonide, budipine, bufexamac, bumetanide, buprenorphine, bupropion, butizine, calcitonin, calcium antagonists, calcium salts, candesartan, capecitabine, captopril, carbamazepine, carifenacin, carvedilol, caspofungin, cefaclor, cefadroxil, cefalexin cefalosporins, cefditoren, cefprozil, cefuroxime, celecoxib, cepecitabine, cerivastatim, cetirizine, cetrorelix, cetuximab, chenodeoxycholic acid, chorionic gonadotropin, ciclosporin, cidofovir, cimetidine, ciprofloxacin, cisplatin, cladribine, clarithromycin, clavulanic acid, clindamycin, clobutinol, clonidine, clopidogrel, codeine, caffeine, colestyramine, cromoglicic acid, cotrimoxazole, coumarin and coumarin derivatives, darbepoetin, cysteamine, cysteine, cytarabine, cyclophosphamide, cyproterone, cytarabine, daclizumab, dalfopristin, danaparoid, dapiprazole, darbepoetin, defepriprone, desipramine, desirudin, desloaratadine, desmopressin, desogestrel, desonide, dexibuprofen, dexketoprofen, disoproxil, diazepam and diazepam derivatives, didanosine, dihydralazine, diltiazem, dimenhydrinate, dimethyl sulfoxide, dimeticone, dipivoxil, dipyridarnoi, dolasetron, domperidone, and domperidane derivatives, donepzil, dopamine, doxazosin, doxorubizin, doxylamine, diclofenac, divalproex, dronabinol, drospirenone, drotrecogin alpha, dutasteride, ebastine, econazole, efavirenz, eletripan, emidastine, emtricitabine, enalapril, encepur, entacapone, enfurvirtide, ephedrine, epinephrine, eplerenone, epoetin and epoetin derivatives, eprosartan, eptifibatide, ertapenem, esomeprazole, estrogen and estrogen derivatives, etanercept, ethenzamide, ethinestradiol, etofenamate, etofibrate, etofylline, etonogestrel, etoposide, exemestan, exetimib, famciclovir, famotidine, faropenan daloxate, felodipine, fenofibrate, fentanyl, fenticonazole, fexofenadine, finasteride, fluconazole, fludarabine, flunarizine, fluorouracil, fluoxetine, flurbiprofen, flupirtine, flutamide, fluvastatin, follitropin, fomivirsen, fondaparinux, formoterol, fosfomicin, frovatriptan, furosemide, fusidic acid, gadobenate, galantamine, gallopamil, ganciclovir, ganirelix, gatifloxacin, gefitinib, gemfibrozil, gemopatrilate, gentamicin, gepirone, progestogen and progestogen derivatives, ginkgo, glatiramer, glibenclamide, glipizide, glucagon, glucitol and glucitol derivatives, glucosamine and glucosamine derivatives, glycoside antibiotics, glutathione, glycerol and glycerol derivatives, hypothalamus hormones, goserelin, grepafloxacin, gyrase inhibitors, guanethidine, gyrase inhibitors, hemin, halofantrine, haloperidol, urea derivatives as oral antidiabetics, heparin and heparin derivatives, cardiac glycosides, hyaluronic acid, hydralazine, hydrochlorothiazide and hydrochlorothiazide derivatives, hydroxyomeprazole, hydroxyzine, ibritumomab, ibuprofen, idarubicin, ifliximab, ifosfamide, iloprost, imatinib, imidapril, imiglucerase, imipramine, imiquimod, imidapril, indometacin, indoramine, infliximab, insulin, insulin glargin, interferons, irbesartan, irinotecan, isoconazole, isoprenaline, itraconazole, ivabradines, iodine and iodine derivatives, St. John's wort, potassium salts, ketoconazole, ketoprofen, ketotifen, lacidipine, lamotrigine, lansoprazole, laronidase, latanoprost, leflunomide, leminoprazole, lepirudin, lercanidipine, leteprinim, letrozole, levacetylmethadol, levetiracetam, levocetirizine, levodopa, levodrpropicin, levofloxacin, levomethadone, licofelone, linezolide, lipinavir, lipoic acid and lipoic acid derivatives, lisinopril, lisuride, lofepramine, lodoxamide, lomefloxacin, lomustine, loperamide, lopinavir, loratadine, lornoxicam, losartan, lumefantrine, lutropine, magnesium salts, macrolide antibiotics, mangafodipir, maprotiline, mebendazole, mebeverine, meclozine, mefenamic acid, mefloquine, meloxicam, memantine, mepindolol, meprobamate, meropenem, mesalazine, mesuximide, metamizole, metformin, methadone, methotrexate, methyl (5-amino-4-oxopentanoate), methylnaloxone, methylnaltrexones, methylphenidate, methylprednisolone, metixen, metoclopramide, metoprolol, metronidazole, mianserin, mibefradil, miconazole, mifepristone, miglitol, miglustad, milnacipran, minocycline, minoxidil, misoprostol, mitomycin, mizolastine, modafinil, moexipril, montelukast, moroctocog, morphinans, morphine and morphine derivatives, moxifloxacin, ergot alkaloids, nalbuphine, naloxone, naproxen, naratriptan, narcotine, natamycin, nateglinide, nebivolol, nefazodone, nelfinavir, neostigmine, neramexan, nevirapine, nicergoline, nicethamide, nifedipine, niflumic acid, nimodipine, nimorazole, nimustine, nesiritide, nisoldipine, norfloxacin, novamine sulfone, noscapine, nystatin, ofloxacin, oktotride, olanzapine, olmesartan, olsalazine, oseltamivir, omapatrilate, omeprazole, omoconazole, ondansetron, orlistat, oseltamivir, oxaceprol, oxacillin, oxaliplatin, oxaprozin, oxcarbacepin, oxicodone, oxiconazole, oxymetazoline, palivizumab, palanosetron, pantoprazole, paracetamol, parecoxib, paroxetine, pegaspargase, peginterferon, pegfilgrastrim, penciclovir, oral penicillins, pentazocine, pentifylline, pentoxifylline, peptide antibiotics, perindopril, perphenazine, pethidine, plant extracts, phenazone, pheniramine, phenylbutyric acid, phenytoin, phenothiazines, phenserine, phenylbutazone, phenytoin, pimecrolimus, pimozide, pindolol, pioglitazone, piperazine, piracetam, pirenzepine, piribedil, pirlindol, piroxicam, posaconazole, pramipexol, pramlintide, pravastatin, prazosin, procaine, promazine, propiverine, propranolol, propionic acid derivatives, propyphenazone, prostaglandins, protionamide, proxyphylline, quetiapine, quinapril, quinaprilate, quinupristine, ramipril, ranitidine, rabeprazole, raloxifen, ranolazine, rasburicase, reboxetin, repaclinides, reproterol, reserpine, revofloxacin, ribavirin, rifampicin, riluzoles, rimexolone, risedronate, risperidone, ritonavir, rituximab, rivastimen, risatriptan, rofecoxib, ropinirol, ropivacaine, rosiglitazone, rotigotine, roxatidine, roxithromycin, ruscogenin, rosuvastatin, rutoside and rutoside derivatives, sabadilla, salbutamol, salicylates, salmeterol, saperconazoles, thyroid hormones, scopolamine, selegiline, sertaconazole, sertindole, sertraline, sevelamer, sibutramine, sildenafil, silicates, simvastatin, sirolimus, sitosterol, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiam, sumatriptan, suxamethonium chloride, tacrine, tacrolimus, tadalafil, taliolol, talsaclidine, tamoxifen, tamsulosin, tasonermin, tazarotene, tegafur, tegaserod, telithromycin, telmisartan, temoporfin, temozolomide, tenatoprazole, tenecteplase, teniposide, tenofovir, tenoxicam, teriparatide, terazosin, terbinafine, terbutaline, terfenadine, teriparatide, terlipressin, tertatolol, testosterone and testosterone derivatives, tetracyclines, tetryzoline, tezosentan, theobromine, theophylline, theophylline derivatives, thiamazole, thiotepa, thr. growth factors, tiagabine, tiapride, tibolone, ticlopidine, tilidine, timolol, tinidazole, tioconazole, tioguanine, tiotropium, tioxolone, tirazetam, tiropramide, trofiban, tizanidine, tolazoline, tolbutamide, tolcapone, tolnaftate, tolperisone, tolterodine, topiramate, topotecan, torasemide, tramadol, tramazoline, trandolapril, tranylcypromine, trapidil, trastuzumab, travoprost, trazodone, trepostinil, triamcinolone and triamcinolone derivatives, triamterene, trifluperidol, trifluridine, trimetazidines, trimethoprim, trimipramine, tripelennamine, triprolidine, trifosfamide, tromantadine, trometamol, tropalpine, trovafloxacin, troxerutin, tulobuterol, trypsins, tyramine, tyrothricin, urapidil, ursodeoxycholic acid, theophylline ursodeoxycholic acid, valaciclovir, valdecoxib, valganciclovir, valproic acid, valsartan, vancomycin, vardenafil, vecuronium chloride, venlafaxine, verapamil, verteporfin, vidarabine, vigabatrine, viloxazine, vinblastine, vincamine, vincristine, vindesine, vinorelbine, vinpocetine, viquidil, vitamin D and derivatives of vitamin D, voriconazole, warfarin, xantinol nicotinate, ximelagatran, xipamide, zafirlukast, zalcitabine, zaleplon, zanamivir, zidovudine, ziprasidone, zoledronic acid, zolmitriptan, zolpidem, zoplicone, zotepine and the like.

Particularly Preferred Active Ingredients

Preferred groups of active ingredients are analgesics, antibiotics, antidiabetics, antibodies, peptides, proteins, chemotherapeutics, corticoids/corticosteroids antiinflammatory agents, enzyme products hormones and their inhibitors, parathyroid hormones digestion-promoting agents, laxatives, vitamins, cytostatics and active ingredients of other groups which, for kinetic reasons, are advantageously administered in lower sections of the intestine.

Examples of particularly preferred active ingredients are mesalazine, sulfasalazine, bethamethasone 21-dihydrogenphosphate, hydrocortisone 21-acetate, cromoglicic acid, dexamethasone, olsalazine Na, budesonide, prednisone bismunitrate, karaya gum, methylprednisolone 21-hydrogensuccinate myhrr, coffee charcoal, chamomile flower extract, preparations of human placenta.

Newer Active Ingredients can be Found from the Literature or from Relevant Pharmaceutical Databases Known to the Person Skilled in the Art:

Balsalazide, adalimumab, alemtuzumab, basiliximab, daclizumab, ibritumomab, ifliximab, cetuximab, palivizumab, rituximab, trastuzumab, other orally administered peptides (e.g. RDP 58), interleukin 6, interleukin 12, ilodecakin (interleukin 10), nicotine tartrate, 5-ASA conjugates (CPR 2015), monoclonal antibodies against interleukin 12, diethyldihydroxyhomospermine (DEHOHO), diethylhomo-spermine (DEHOP), cholocystokinin (CCK) antagonist (CR 1795), 15 amino acid fragment of a 40 kd peptide from gastric juice (BPC 15), glucocorticoid analog (CBP 1011), natalizumab, infliximab (REMICADE) N-deacetylated lysoglycosphingolipid (WILD 20), azelastine, tranilast, sudismase, phosphorothioate antisense oligonucleotide (ISIS 2302), tazofelone ropivacaine, 5 lipoxygenase inhibitor (A 69412), sucralfate

The active ingredients may if desired also be used in the form of their pharmaceutically acceptable salts or derivatives, and in the case of chiral active ingredients it is possible to employ both optically active isomers and racemates or mixtures of diastereomers. The active ingredients may likewise be in the form of physical or chemical conjugates (polymer-drug conjugates, e.g. peptide/protein-active ingredient complexes). If desired, the compositions of the invention may also comprise two or more active pharmaceutical ingredients.

### EXAMPLES

Description of Experiments

Items of Apparatus

Hüttlin Mycrolab fluidized bed apparatus

Nozzle: three-fluid nozzle, nozzle diameter: 0.8 mm

Method: bottom spray

Peristaltic pump: Ismatec MCP

Coatings

Material

Theophylline pellets (particle diameter: 0.8-1.2 mm)

Active ingredient content: about 93%

Batch size: 200 or 800 g

Coating Conditions

Inlet temperature: 33-43° C.

Process temperature: 25-31° C.

Spraying pressure: 0.6-0.75 bar

Microclimate: 0.4-0.5 bar

Spraying rate: for 200 g batch size: about 12 g/min/kg
for 800 g batch size: about 5 g/min/kg

Samples taken at 6 and 10% polymer application.

Polymers

Polymer Type (I)

Eudragit® FS 30 D (FS30 D):

Methyl acrylate methyl methacrylate methacrylic acid copolymer

Polymer Types (II)

Eudragit® NE 30 D (NE30 D):

Ethyl acrylate methyl methacrylate copolymer

Kollicoat® SR 30 D:

Polyvinyl acetate

Aquacoat® ECD:

Ethylcelluose polymer

(All 30% strength aqueous dispersions)

Plasticizer: DBS=dibutyl sebacate

Mixtures

| | | | | | | | | | Plasti- |
|---|---|---|---|---|---|---|---|---|---|
| Polymer | Proportion | | | | | | | | cizer |
| Eudragit ® | 100 | 5 | 10 | 20 | 50 | — | 10 | 10 | — |
| FS 30 D | | | | | | | | | |
| Eudragit ® | — | 95 | 90 | 80 | 50 | 100 | — | — | — |
| NE 30 D | | | | | | | | | |
| Kollicoat ® | — | — | — | — | — | — | 90 | — | — |
| SR 30 D | | | | | | | | | |
| Aquacoat ® | — | — | — | — | — | — | — | 90 | 24% |
| ECD | | | | | | | | | DBS |
| | | | | | | | | | based |
| | | | | | | | | | on |
| | | | | | | | | | polym. |

Formulation

### EXAMPLES

Spray suspension for 800 g of pellets and 15% polymer application rate:

| | | | Proportions |
|---|---|---|---|
| | Suspension [g] | Solid [g] | [%] |
| Polymer mixture | 400 | 120 | 93.4 |
| Glycerol monostearate | 6 | 6 | 4.7 |
| Polysorbate 80 | 7.3 | 2.4 | 1.9 |
| Deionized water | 228.7 | — | — |
| | 642.0 | 128.4 | 100 |

DM content of spray suspension: 20.0%

Preparation of the Spray Suspension:

Deionized water and polysorbate 80 are heated with gentle stirring to 75° C. The glycerol monostearate is added thereto and homogenized while stirring vigorously for about 30 minutes. Cooling to room temperature is followed by addition of the polymer dispersions and of the plasticizer. If necessary, coagulation on mixing the dispersions is prevented by previous equalization of the pH values.

Release of Active Ingredient (Tables)

USP Release Test

The release test complies with USP 28-NF23, General Chapter <711>, Dissolution, Apparatus 2, (Paddle), Method <724> “Delayed Release (Enteric Coated) Articles-General General Drug Release Standard”, Method B (100 rpm, 37° C.) with the following modification: the coated pellets were initially tested in simulated gastric fluid (USP) at pH 1.2 for resistance to gastric fluid for 120 min, and then the buffer was changed to phosphate buffer of pH 7.5, equivalent to a simulated intestinal environment. The active ingredient concentration in the test medium was determined by photometry.

The release of active ingredient after 120 min should not exceed about 5%. After 180 min, corresponding to 60 min at pH 7.5, the desired degree of release of active ingredient is from 5 to 95%, preferably from 10 to 50%.

The results are compiled in Tables 1 to 3. The statement 6, 10 and 15% indicates in each case the dry weight of the coating based on the weight of the core.

TABLE 1

| | Release of active ingredients [%] | | | | | | | | |
|---|---|---|---|---|---|---|---|---|---|
| | | | | FS 30 D/NE 30 D | | | FS 30 D/NE 30 D | | |
| | NE 30 D | | | 5:95 | | | 10:90 | | |
| | not according to | | | according to the | | | according to the | | |
| Time | the invention | | | invention | | | invention | | |
| [min] | 6% | 10% | 15% | 6% | 10% | 15% | 6% | 10% | 15% |
| 0 | 0.00 | 0.00 | 0.00 | 0.00 | 0.00 | 0.00 | 0.01 | 0.00 | 0.00 |
| 5 | 0.02 | 0.00 | 0.00 | 0.05 | 0.01 | 0.02 | 0.19 | 0.01 | 0.01 |
| 30 | 0.21 | 0.04 | 0.03 | 0.45 | 0.11 | 0.08 | 0.56 | 0.17 | 0.04 |
| 60 | 0.62 | 0.11 | 0.04 | 1.25 | 0.28 | 0.22 | 1.25 | 0.59 | 0.09 |
| 90 | 1.16 | 0.18 | 0.06 | 2.48 | 0.51 | 0.38 | 1.95 | 1.13 | 0.16 |
| 120 | 1.75 | 0.29 | 0.09 | 5.94 | 0.78 | 0.56 | 2.70 | 1.71 | 0.25 |
| 140 | 6.51 | 5.22 | 0.42 | 20.23 | 6.73 | 1.49 | 9.47 | 6.77 | 1.23 |
| 150 | 6.70 | 5.26 | 0.43 | 23.70 | 8.24 | 2.30 | 14.31 | 10.87 | 2.66 |
| 165 | 6.99 | 5.31 | 0.45 | 20.73 | 10.63 | 3.80 | 21.69 | 16.96 | 5.66 |
| 180 | 7.28 | 5.38 | 0.47 | 25.60 | 13.00 | 5.42 | 29.11 | 22.89 | 9.31 |
| 210 | 7.89 | 5.51 | 0.50 | 34.97 | 17.60 | 8.88 | 43.13 | 34.32 | 17.33 |
| 240 | 8.53 | 5.65 | 0.54 | 44.66 | 21.96 | 12.49 | 54.07 | 45.24 | 24.44 |
| 300 | 9.89 | 5.99 | 0.63 | 62.16 | 30.26 | 19.70 | 65.34 | 65.78 | 36.86 |
| 330 | 10.65 | 6.17 | 0.69 | 70.30 | 34.25 | 23.13 | 68.94 | 75.16 | 42.61 |

TABLE 2

| | Release of active ingredients [%] | | | | | | | | |
|---|---|---|---|---|---|---|---|---|---|
| | FS 30 D/NE 30 D | | | FS 30 D/NE 30 D | | | | | |
| | 20:80 | | | 50:50 | | | FS 30 D | | |
| | not according to | | | not according to | | | not according to | | |
| | the invention | | | the invention | | | the invention | | |
| Time [min] | 6% | 10% | 15% | 6% | 10% | 15% | 6% | 10% | 15% |
| 0 | 0.00 | 0.00 | 0.00 | 0.00 | 0.00 | 0.00 | 0.01 | 0.00 | 0.00 |
| 5 | 0.01 | 0.01 | 0.01 | 0.00 | 0.00 | 0.00 | 0.08 | 0.01 | 0.00 |
| 30 | 0.44 | 0.11 | 0.05 | 0.43 | 0.08 | 0.01 | 0.65 | 0.05 | 0.02 |
| 60 | 1.55 | 0.50 | 0.13 | 1.84 | 0.60 | 0.10 | 1.56 | 0.12 | 0.03 |
| 90 | 2.75 | 0.98 | 0.25 | 3.42 | 1.28 | 0.27 | 2.68 | 0.19 | 0.04 |
| 120 | 3.97 | 1.50 | 0.39 | 5.00 | 1.99 | 0.45 | 3.83 | 0.28 | 0.05 |
| 140 | 45.76 | 34.02 | 22.62 | 51.47 | 44.55 | 25.86 | 100.06 | 100.22 | 98.55 |
| 150 | 64.35 | 48.38 | 36.39 | 70.45 | 63.14 | 44.27 | 99.73 | 99.75 | 99.81 |
| 165 | 84.84 | 66.84 | 53.92 | 87.02 | 82.91 | 66.37 | 99.75 | 99.90 | 99.85 |
| 180 | 94.87 | 81.98 | 69.14 | 94.01 | 92.57 | 81.57 | 99.75 | 99.95 | 99.76 |
| 210 | 97.49 | 97.10 | 91.23 | 96.70 | 99.40 | 97.02 | 99.78 | 99.90 | 99.85 |
| 240 | 97.76 | 98.85 | 97.48 | 96.79 | 99.86 | 99.25 | 99.88 | 99.97 | 99.89 |
| 300 | 97.90 | 99.48 | 99.72 | 96.99 | 99.72 | 99.33 | 100.08 | 100.15 | 99.99 |
| 330 | 98.03 | 99.50 | 99.81 | 97.11 | 99.77 | 99.49 | 100.15 | 100.11 | 100.08 |

TABLE 3

| Release of active ingredients [%] | |
|---|---|
| FS 30 D/ | FS 30 D/ |
| Kollicoat ® SR 30 D | Aquacoat ® ECD |
| 10:90 | 10:90 |
| according to the | according to the |
| invention | invention |

| Time [min] | 6% | 10% | 15% | 6% | 10% | 15% |
|---|---|---|---|---|---|---|
| 0 | 0.00 | 0.00 | 0.00 | 0.00 | 0.01 | 0.01 |
| 5 | 0.18 | 0.05 | 0.02 | 0.14 | 0.07 | 0.09 |
| 30 | 1.52 | 0.31 | 0.09 | 1.11 | 0.63 | 0.78 |
| 60 | 3.15 | 0.66 | 0.19 | 2.38 | 1.22 | 1.39 |
| 90 | 4.85 | 1.15 | 0.28 | 3.64 | 1.81 | 2.00 |
| 120 | 6.60 | 1.73 | 0.39 | 5.10 | 2.41 | 2.59 |
| 140 | 13.97 | 2.92 | 0.80 | 5.72 | 2.59 | 2.73 |
| 150 | 22.03 | 6.07 | 0.87 | 6.21 | 2.79 | 2.90 |
| 165 | 35.45 | 13.90 | 1.04 | 7.01 | 3.10 | 3.19 |
| 180 | 49.02 | 23.37 | 1.50 | 7.87 | 3.44 | 3.50 |
| 210 | 73.65 | 43.52 | 6.99 | 9.76 | 4.26 | 4.19 |
| 240 | 91.51 | 63.15 | 18.88 | 11.93 | 5.26 | 5.00 |
| 300 | 99.94 | 93.76 | 49.70 | 16.95 | 7.91 | 7.24 |
| 330 | 100.06 | 98.40 | 65.07 | 19.63 | 9.51 | 8.66 |

1-10. (canceled)

11. A pharmaceutical form comprising an active ingredient-containing core which is coated with a mixed polymeric coating, characterized in that said mixed coating is a mixture of 2 to 60% by weight of one or more polymers (I) with 40 to 98% by weight of one or more polymers (II), wherein
polymer (I) is a copolymer of 10 to 30% by weight methyl methacrylate, 50 to 70% by weight methyl acrylate and 5 to 15% by weight methacrylic acid, and
polymer (II) is a vinyl polymer different from polymer (I) or a polysaccharide or a derivative of a polysaccharide which is composed to the extent of 88 to 100% of neutral monomer units and may comprise up to 12% by weight monomer units having ionic radicals.

12. The pharmaceutical form as claimed in claim 11, wherein polymer (II) is a copolymer of methyl methacrylate and ethyl acrylate, a copolymer of methyl methacrylate and ethyl acrylate and methacrylic acid, a copolymer of methyl methacrylate, ethyl acrylate and trimethylammoniumethyl methacrylate, polyvinylpyrrolidones (PVP), polyvinyl alcohols, a polyvinyl alcohol-polyethylene glycol graft copolymer, starch and derivatives thereof, polyvinyl acetate (PVAc), a vinyl acetate-vinylpyrrolidone copolymer, hydroxyethylcellulose (HEC), hydroxypropylcellulose (HPC), hydroxypropylmethylcellulose (HPMC), hydroxymethylethylcellulose (HEMC), ethylcellulose (EC), methylcellulose (MC), cellulose esters, cellulose glycolate or a mixture of said polymers.

13. The pharmaceutical form as claimed in claim 12, wherein polymer (II) is a copolymer of 20 to 40% by weight ethyl acrylate and 60 to 80% by weight methyl methacrylate.

14. The pharmaceutical form as claimed in claim 12, wherein polymer (II) is a copolymer of 50-70% by weight methyl methacrylate, 20-40% by weight ethyl acrylate and 12-2 by weight trimethylammoniumethyl methacrylate chloride.

15. The pharmaceutical form as claimed in claim 11, characterized in that wherein the polymer coating amounts to 2 to 20% by weight in relation to the weight of the active ingredient-containing core.

16. The pharmaceutical form as claimed in claim 11, wherein the release of active ingredient at the pH at which polymer (I) starts to dissolve, in the USP release test, is less than 50% in 60 minutes.

17. The pharmaceutical form as claimed in claim 11, wherein said form is multiparticulate pharmaceutical form, selected from a pellet-containing tablet, minitablet, capsule, sachet or reconstitutable powder.

18. A method for the production of a coated pharmaceutical form comprising an active ingredient-containing core and a polymeric coating of a mixture of polymers, said mixture comprising 2 to 60% by weight of one or more polymers (I) with 40 to 98% by weight of one or more polymers (II), wherein
polymer (I) is a (meth)acrylate copolymer comprising 90 to 100% by weight free radically polymerized units of 40 to 95% by weight of C₁- to C₄-alkyl esters of acrylic or methacrylic acid and 5 to 60% by weight units of (meth)acrylate monomers having an anionic group, and 0 to 10% by weight of additional polymerizable vinyl monomers, and
polymer (II) is a vinyl polymer different from polymer (I) or a polysaccharide or a derivative of a polysaccharide comprising 88 to 100% neutral monomer units and up to 12% by weight polymerized monomer units having ionic radicals,
characterized in that
the glass transition temperature of polymer (I) is not more than 70° C., and the active ingredient is released more slowly by comparison with a pharmaceutical form coated with polymer (I) alone starting at the same pH.

19. The method as claimed in claim 18, characterized in that polymer (I) is a copolymer of 10 to 30% by weight methyl methacrylate, 50 to 70% by weight methyl acrylate and 5 to 15% by weight methacrylic acid.

20. The method as claimed in claim 18, characterized in that polymer (I) is a copolymer which is composed of
20 to 34% by weight methacrylic acid and/or acrylic acid,
20 to 69% by weight methyl acrylate,
0 to 40% by weight ethyl acrylate and/or optionally
0 to 10% by weight additional copolymerizable vinyl monomers,
with the proviso that the glass transition temperature of the copolymer does not exceed 60° C.

21. The method as claimed in claim 18, characterized in that polymer (I) is a copolymer which is composed of
20 to 33% by weight methacrylic acid and/or acrylic acid,
5 to 30% by weight methyl acrylate,
20 to 40% by weight ethyl acrylate,
more than 10 to 30% by weight butyl methacrylate,
and optionally
0 to 10% by weight additional copolymerizable vinyl monomers, where the proportions of the monomers add up to 100% by weight,
with the proviso that the glass transition temperature of the copolymer is 55 to 70° C.

22. The method as claimed in claim 18, wherein polymer (II) is a copolymer of methyl methacrylate and ethyl acrylate, a copolymer of methyl methacrylate and ethyl acrylate and methacrylic acid, a copolymer of methyl methacrylate, ethyl acrylate and trimethylammoniumethyl methacrylate, polyvinylpyrrolidones (PVP), polyvinyl alcohols, polyvinyl alcohol-polyethylene glycol graft copolymer, starch and derivatives thereof, polyvinyl acetate (PVAc), a vinyl acetate-vinylpyrrolidone copolymer, hydroxyethylcellulose (HEC), hydroxypropylcellulose (HPC), hydroxypropylmethylcellulose (HPMC), hydroxymethylethylcellulose (HEMC), ethylcellulose (EC), methylcellulose (MC), cellulose esters, cellulose glycolate or a mixture of said polymers.

23. The method as claimed in claim 22, wherein polymer (II) is a copolymer of 20 to 40% by weight ethyl acrylate and 60 to 80% by weight methyl methacrylate.

24. The use as claimed in claim 22, wherein polymer (II) is a copolymer of 50-70% by weight methyl methacrylate, 20-40% by weight ethyl acrylate and 12-2 by weight trimethylammoniumethyl methacrylate chloride.

25. The method as claimed in claim 22, wherein the polymer coating amounts to 2 to 20% by weight in relation to the weight of the active ingredient-containing core.

26. The method as claimed in claim 22, wherein the release of active ingredient at the pH at which polymer (I) starts to dissolve, in the USP release test, is less than 50% in 60 minutes.

27. The method as claimed in claim 22, wherein the coated pharmaceutical form is in the form of pellets which are present in a multiparticulate pharmaceutical form selected from pellet-containing tablets, minitablets, capsules, sachets or reconstitutable powders.
